(19) United States
(12) Patent Application Publication
(10) Pub. No.: US 2019/0382327 A1
(43) Pub. Date: Dec. 19, 2019

(54) METHOD AND APPARATUS FOR PRODUCING CRYSTALLINE MATERIAL FROM PLANT EXTRACT

(71) Applicants: Jacqueline McGrane, Black Hawk, CO (US); Randall Davis, Thornton, CO (US)

(72) Inventors: Jacqueline McGrane, Black Hawk, CO (US); Randall Davis, Thornton, CO (US)

(21) Appl. No.: 16/445,151

(22) Filed: Jun. 18, 2019

Related U.S. Application Data
(60) Provisional application No. 62/686,422, filed on Jun. 18, 2018.

(51) Int. Cl. C07C 37/84; C11B 3/10; B01D 9/00

(52) U.S. Cl. CPC C07C 37/84; C07C 39/23; B01D 9/00; C11B 3/10

(57) ABSTRACT

Apparatuses and methods for producing crystalline materials from plant extracts are described herein. In one example, an apparatus for producing crystalline material from plant extract can include a vessel comprising an inner volume configured to receive a plant extract. The apparatus can include a desiccant chamber fluidly connected to the vessel. The desiccant chamber can house a desiccant material. The desiccant material can absorb water vapor from the plant extract and thereby promote growth of crystalline material.

### CROSS-REFERENCE TO RELATED APPLICATIONS

This application claims the benefit of U.S. Provisional Patent Application No. 62/686,422, filed on Jun. 18, 2018, which is hereby incorporated by reference in its entirety as if fully set forth in this description.

### FIELD

This disclosure relates to methods and apparatuses for processing biomass extracts. More specifically, this disclosure relates to methods and apparatuses for producing crystalline materials from plant extracts.

### BACKGROUND

Useful plant extracts, such as essential oils, can be extracted from certain plant matter. Examples of plant matter that produce useful extracts include lavender flowers, eucalyptus leaves, peppermint leaves, tea tree leaves, jojoba seeds, rose petals, cannabis flowers and leaves, and jasmine flowers. Plant extracts are used in a wide variety of applications, including as additives in household cleansers and personal care products (e.g. shampoos, lotions, and facial cleansers) and in pain relief treatments.

Crude plant extracts can be further processed to yield more refined products. In one example, crude plant extracts can be processed to yield crystalline materials. Crystalline materials may have desirable attributes, such as being more stable and having longer shelf lives than crude plant extracts.

A known method for producing crystalline material from cannabis extract involves pouring crude oil extracted from cannabis plant material into a glass canning jar. The glass canning jar is then heated to a temperature slightly above room temperature to promote vaporization and off-gassing of any solvent remaining in the extract following a solvent-based extraction process. Because the solvent is a flammable hydrocarbon gas, it is desirable to remove as much of the solvent as possible before sealing the jar. After a lid is sealed on the canning jar, the canning jar is placed in a dark location for one or two months. During this period of time, crystalline material may form in the jar. The jar may need to be opened periodically during the process to release pressure resulting from evaporation of remaining solvent. If the jar is not opened during the process, or is not opened frequently enough, pressure within the jar may exceed structural limits of the glass jar, resulting in failure of the jar and an explosion that may harm people and/or damage property. In addition to being unsafe, this method requires a long duration to produce crystalline material (e.g. 1-2 months). An improved method and apparatus are needed.

### BRIEF DESCRIPTION OF THE DRAWINGS

FIG. 1 shows a front perspective view of an apparatus for producing crystalline material from plant extract or distillate.

FIG. 2 shows a side perspective view of the apparatus of FIG. 1.

FIG. 3 shows an exploded view of the apparatus of FIG. 1.

FIG. 4 shows an operator installing a bottom wall on a vessel.

FIG. 5 shows a clamp and stand installed on a bottom side of the vessel.

FIG. 6 shows a desiccant chamber containing desiccant material.

FIG. 7 shows the desiccant chamber of FIG. 6 with a screen installed over an opening in the chamber.

FIG. 8 shows the desiccant chamber of FIG. 6 attached to a lid assembly.

FIG. 9 shows a viscous plant extract being poured into the vessel.

FIG. 10 shows an operator measuring hydrocarbon levels with a hydrocarbon meter near an upper opening of the vessel.

FIG. 11 shows an operator securing the lid assembly on the vessel to seal the apparatus of FIG. 1.

FIG. 12 shows a supply tank of pressurized gas.

FIG. 13 shows a supply line from the supply tank of FIG. 12 attached to a gas input on the lid assembly of the apparatus.

FIG. 14 shows the apparatus in an oven.

FIG. 15 shows a side window of the vessel containing High Terpene Full Spectrum Extract and High Cannabinoid Full Spectrum Extract.

FIG. 16 shows the vessel upside down and draining HTFSE into a container after HTFSE has been poured from the vessel into an adjacent container.

FIG. 17 shows HCFSE in a bottom portion of the vessel.

FIG. 18 shows HCFSE produced by the apparatus.

FIG. 19 shows THCA sugar produced by the apparatus.

FIG. 20 shows CBD crystals produced by the apparatus.

### BRIEF SUMMARY

The apparatuses and methods disclosed herein can safely produce crystalline materials from plant extracts. The apparatuses and methods described herein may produce crystalline materials in significantly less time than existing methods and apparatuses. The apparatuses and methods described herein may produce higher quality crystalline materials than existing methods and apparatuses.

Plant extracts made from fresh cannabis material may contain a significant amount of water. Water may slow or inhibit growth of crystalline material. It is therefore desirable to provide an apparatus that is capable of removing water from the plant extract during the crystallization process. The apparatus described herein may include a desiccant material that removes water from the plant extract after the vessel has been sealed. By removing water from the plant extract, the apparatus may increase the rate of crystal growth and the quality of crystals grown. The result may be large, dense crystal clusters that cannot be produced with an apparatus that lacks a desiccant chamber.

In one example, an apparatus for producing crystalline material from plant extract can include a vessel. The vessel may have an inner volume defined by one or more inner surfaces. The inner volume may be configured to receive a plant extract. The apparatus may include a desiccant chamber fluidly connected to the vessel. The desiccant chamber may be configured to receive a desiccant material. The apparatus may include a fluid pathway extending from the inner volume of the vessel to the desiccant chamber. The apparatus may include a valve located in the fluid pathway. The valve may have an open position and a closed position, and water vapor from the plant extract can travel through the fluid pathway from the inner volume to the desiccant chamber when the valve is in the open position. The desiccant material in the desiccant chamber may absorb the water vapor from the plant extract, thereby promoting growth of crystalline material within the vessel.

### DETAILED DESCRIPTION

The apparatus described herein is configured to receive a source material and output a useful product. In one example, the source material may be a plant extract, such as a cannabis extract, and the product may be a crystalline material. The apparatus may be capable of receiving a variety of source materials and outputting a variety of useful products, as described herein.

To obtain a suitable source material, an extraction process may be utilized to extract useful components from plant material. In one example, an extraction process can be a solvent-based extraction process. The solvent can be any suitable solvent, such as butane, propane, or ethanol. When cannabis is subjected to a solvent-based extraction process, the process may produce a cannabis extract. When fresh cannabis or fresh, frozen cannabis is subjected to an extraction process, the resulting extract may be referred to as live resin. When dried cannabis is subjected to an extraction process, the resulting extract may be referred to as crude oil.

Live resin can be produced by freezing fresh cannabis flowers (e.g. with liquid nitrogen or dry ice) and then performing an extraction process on the frozen plant material using propane and/or butane.

Fresh cannabis contains higher water content than dried and cured cannabis plant material. Since butane is water soluble above certain temperatures, to prevent contamination when butane is used as the solvent, live resin may be extracted at very low temperatures. For example, extractions of fresh, frozen plant matter may occur between −50 and −60 degrees Fahrenheit or lower to prevent toxins from the butane from polluting the extract as the frozen cannabis plant is washed in the solvent.

During the extraction process, solvent may wash and/or dissolve cannabinoids and terpenes from the plant material, resulting in a mixture containing solvent, cannabinoids, and terpenes. The bulk of the solvent can then be purged from the mixture to yield a cannabis concentrate containing cannabinoids, terpenes, and some solvent.

The resulting live resin can then be used as a source material for the apparatuses and methods described herein. If fresh cannabis flowers are not available, dried cannabis plant material can be substituted but will yield different products, such as THC sugar, as described herein. Due to volatility of terpenes, dried cannabis flowers commonly contain fewer terpenes than fresh cannabis flowers. Consequently, crystalline products made from extracts created from dried cannabis material typically contain fewer terpenes than crystalline products made with live resin.

The cannabis extract may be a viscous extraction containing a small amount of solvent. When butane is used in the solvent-extraction process, the resulting viscous cannabis extract may be referred to as butane hash oil (BHO). After the extraction process is complete, it may be desirable to retain a small amount of solvent in the extract to reduce viscosity and allow the mixture to remain pourable. For example, it may be desirable to retain enough solvent in the extract mixture to maintain a viscosity similar to honey to allow for transfer of the extract to the vessel 105 described herein. FIG. 9 shows an example of pouring the viscous extract mixture into an inner volume of the vessel 105 of the apparatus 100.

When transferred to the apparatus 100 and subjected to the methods described herein, the cannabis extract may transform into a crystalline material. The crystalline material may have desirable attributes. For instance, the crystalline material may have higher purity than the original extract (e.g. live resin or crude oil). The crystalline material may have higher potency than the original extract. The crystalline material may be more stable and have a longer shelf life than the original extract. The crystalline material may be easier to store and transport than the original extract. The crystalline material may be easier to precisely dose and administer than the original extract.

Cannabis extracts, such as live resin, contain water. Water may inhibit growth of crystalline material. Removing water from the cannabis extract during the crystallization process may promote formation of crystalline material. The apparatus 100 described herein can remove water content from the vessel 105 while the vessel remains sealed. The apparatus 100 can remove water content from the vessel 105 while the vessel remains sealed and at a pressure above atmospheric pressure. Capturing water from the cannabis extract using a desiccant material, as described herein, may allow crystals to grow more rapidly than in an apparatus without a desiccant material.

Elevated pressure may promote growth of crystalline material. The apparatus may be capable of being pressurized above atmospheric pressure. For example, the vessel 105 may be pressurized up to about 14 psig. Applying pressure to the extract may increase the rate of crystal growth. Applying pressure to the extract may increase the size of crystals formed. Applying pressure to the extract may increase the quantity of crystals formed. As described herein, the apparatus 100 may include a vessel 105 with an inner geometry that promotes formation of crystalline material. The apparatus 100 may have enhancements, such as nucleation sites, on internal surfaces of the vessel 105 to promote formation of crystalline material.

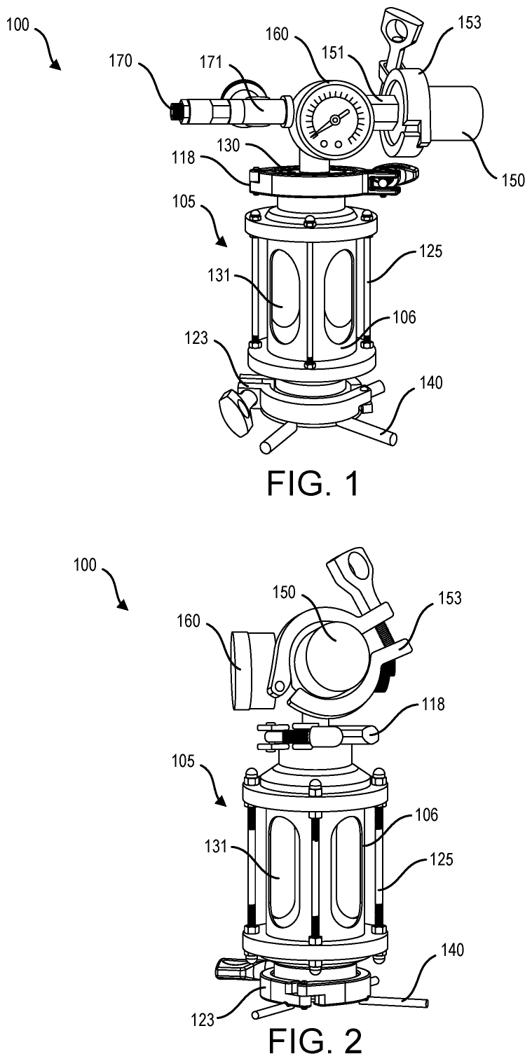

FIGS. 1 and 2 show an apparatus 100 for producing crystalline material (e.g. 192, 500, 600) from a plant extract (e.g. 190). The apparatus 100 may include a vessel 105 for receiving a viscous plant extract 190. The vessel 105 may have an inner volume 110 defined by a plurality of inner surfaces. The vessel 105 may be a pressure vessel. In one example, the vessel 105 may include a bottom wall 107, a top wall 130, and a side wall 106 extending from the bottom wall 107 to the top wall 130. In one example, the vessel 105 may be an opaque chamber constructed of a suitable material, such as stainless steel. In another example, the vessel 105 may include one or more transparent portions allowing an interior volume of the vessel to be viewed from outside the vessel, thereby allowing visual monitoring of vessel contents during a crystallization process. In another example, as shown in FIG. 1, the vessel 105 may be a bolt style sight glass enclosed by a bottom wall 107 and a top wall 130.

The vessel 105 may include an upper flange 116 and a lower flange 121. The vessel 105 may include a glass cylinder 131 located between the upper flange 116 and the lower flange 121, and a plurality of elongated support members 125 extending from the upper flange 116 to the lower flange 121. The glass cylinder 131 may be made of low-thermal-expansion borosilicate glass, such as PYREX. In yet another example, the chamber 105 may be a flange style sight glass.

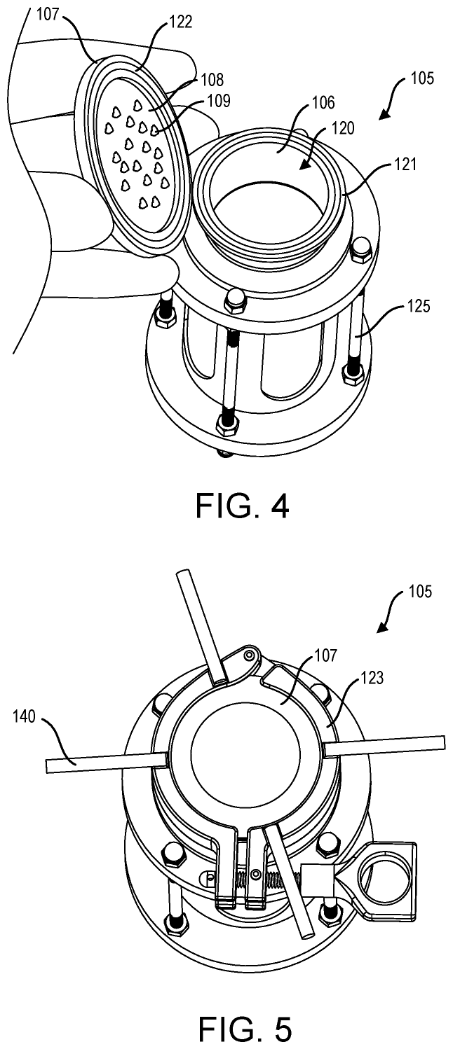

The vessel 105 may include an upper opening 115. The vessel 105 may include a lower opening 120, as shown in FIG. 4. The upper and lower openings (115, 120) may allow access to the inner volume 110 of the vessel 105. The upper and lower openings (115, 120) may be enclosed by additional components, such as those described herein, to seal the vessel 105.

In one example, the lower opening 120 may be enclosed by the bottom wall 107. The bottom wall 107 of the vessel 105 may be an end cap, as shown in FIG. 4. The end cap 107 may be installed to cover and seal the lower opening 120. The end cap 107 may be secured to the lower flange 121 by a lower clamp 123. The lower clamp 123 may be a tri-clamp. A seal 122 may be provided between the end cap 107 and a sealing surface of the lower flange 121. The seal 122 may be a chemical-resistant gasket. In one example, the seal 122 may be a gasket made of a fluoropolymer elastomer, such as VITON. Fluoropolymer elastomers may be chemically-resistant to terpenes and hydrocarbons.

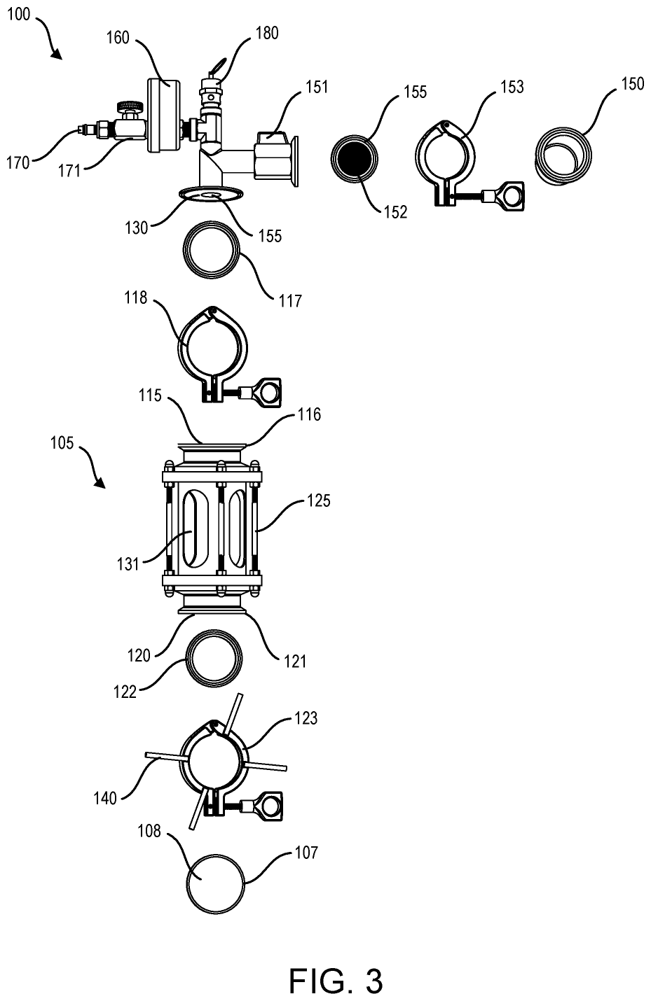

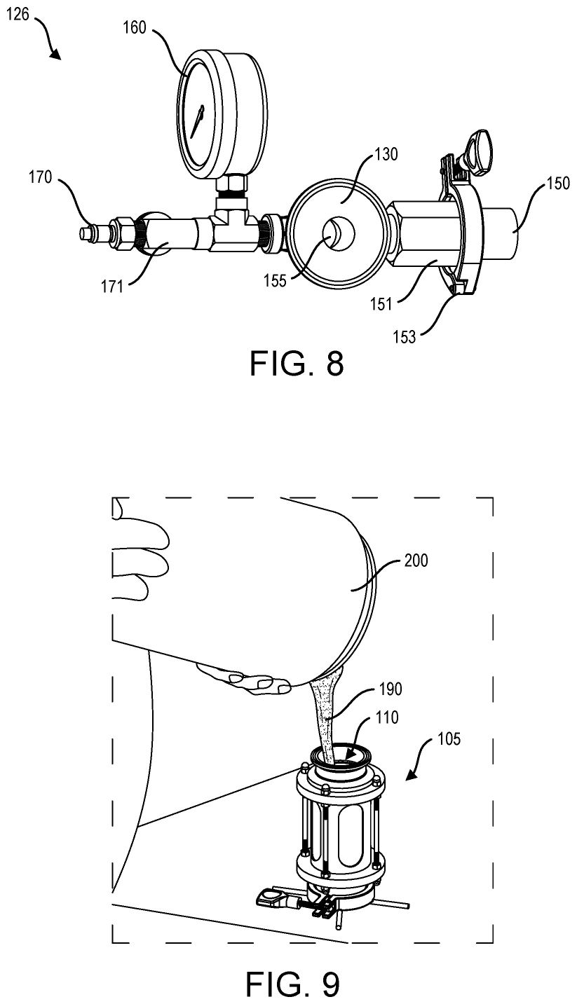

In one example, the upper opening 115 may be enclosed by a top wall 130. The top wall 130 of the vessel 105 may be a lid 130 or a lid assembly 126, as shown in FIGS. 1 and 8. The lid assembly 126 may be installed to cover and seal the upper opening 115. The lid assembly 126 may be secured to the upper flange 116 by an upper clamp 118. As shown in FIG. 3, a seal 117 may be provided between the lid 130 and a sealing surface of the upper flange 116. The seal 117 may be a chemical-resistant gasket. In one example, the seal 117 may be a gasket made of a fluoropolymer elastomer, such as VITON.

An interior surface 108 of the bottom wall 107 may include one or more nucleation sites 109, as shown in FIG. 4. The one or more nucleation sites 109 may promote formation of crystalline material in the vessel 105. The one or more nucleation sites 109 may promote growth of large crystals. In one example, the interior surface 108 of the bottom wall 107 may include a plurality of nucleation sites 109 formed by laser etching. In another example, the interior surface 108 of the bottom wall 107 may be polished stainless steel and may include a plurality of nucleation sites 109 formed by laser etching. The nucleation sites 109 may have an average depth of less than or equal to 0.001 inch. In another example, the interior surface 108 of the bottom wall 107 may be polished stainless steel and include a plurality of nucleation sites 109 formed by laser engraving. The nucleation sites 109 may have an average depth of less than or equal to 0.020 inch. The nucleation sites 109 may have an average depth of less than or equal to 0.010 inch. The nucleation sites 109 may have an average depth of less than or equal to 0.05 inch. The nucleation sites 109 may have an average depth of less than or equal to 0.001 inch. A polished stainless-steel interior surface 108 may ease removal of crystalline product from the vessel when the crystallization process is finished.

The vessel 105 may have a depth (d) and a width (w). The depth may be greater than the width, which may improve performance of the apparatus 100. For instance, providing a vessel 105 with a depth (d) greater than a width (w) may enhance or promote crystal formation during use of the apparatus 100. The depth (d) may be at least two times greater than the width (w). The depth (d) may be at least three times greater than the width (w). The depth (d) may be at least three times greater than the width (w). The depth (d) may be at least five times greater than the width (w). In one example, the vessel 105 may have an inner volume about equal to 300 mL (10.14 oz), an inner width (w) of about 2 inches, and an inner depth (d) of about 3.2 inches. In this example, the depth (d) is about 1.6 times greater than the width (w). In another example, the vessel 105 may have an inner volume about equal to 700 mL (23.67 oz), an inner width (w) of about 3 inches, and an inner depth (d) of about 3.3 inches. In this example, the depth (d) is about 1.1 times greater than the width (w).

The apparatus 100 may include a pressure gauge 160 fluidly connected to the vessel 105. The pressure gauge 160 may measure and display a pressure or otherwise output an electronic signal corresponding to a pressure within the vessel 105. The pressure gauge 160 may allow an operator to accurately raise the pressure within the vessel 105 until a target pressure is reached and confirmed by a visual or electronic output from the pressure gauge 160. After the vessel 105 is pressurized, the pressure gauge 160 may allow an operator to monitor the pressure over time to ensure the vessel 105 is properly sealed and not losing pressure. The pressure gauge 160 may include an alarm if the pressure exceeds a predetermined maximum pressure (e.g. about 6 psig). The pressure gauge 160 may include an alarm if the pressure falls below a predetermined minimum pressure (e.g. about 4 psig).

The apparatus 100 may include a pressure relief valve 180 fluidly connected to the vessel 105, as shown in FIG. 13. For safety, the pressure relief valve 180 may open when the pressure in the vessel 105 exceeds a predetermined pressure. Upon activation, the pressure relief valve 180 may open and allow gas to escape from the vessel 105, thereby reducing pressure within the vessel 105 to a safe level. The pressure relief valve 180 may prevent the vessel 105 from reaching an internal pressure that might otherwise cause failure of the vessel or other components. In one example, the pressure relief valve 180 may be set to open at a pressure at or above 12 psig. In another example, the pressure relief valve 180 may be set to open at a pressure at or above 14 psig. In yet another example, the pressure relief valve 180 may be set to open at a pressure at or above 16 psig.

During use of the apparatus 100, all or a portion of air may be evacuated from the vessel 105 and replaced by a gas that promotes growth of crystalline material. A hydrocarbon gas may be added to the vessel 105 (e.g. through evaporation of a hydrocarbon solvent present in the plant extract that is added to and sealed within the vessel). In one example, propane gas may be added to the vessel 105. In another example, butane gas may be added to the vessel 105. In yet another example, a mixture of propane and butane gas may be added to the vessel 105. An inert gas may be added to the vessel 105. For example, nitrogen may be added to the vessel 105 from an external source. The presence of nitrogen may promote nucleation and accelerate crystal growth. In other examples, air may not be purged from the vessel 105 prior to adding a hydrocarbon gas or nitrogen.

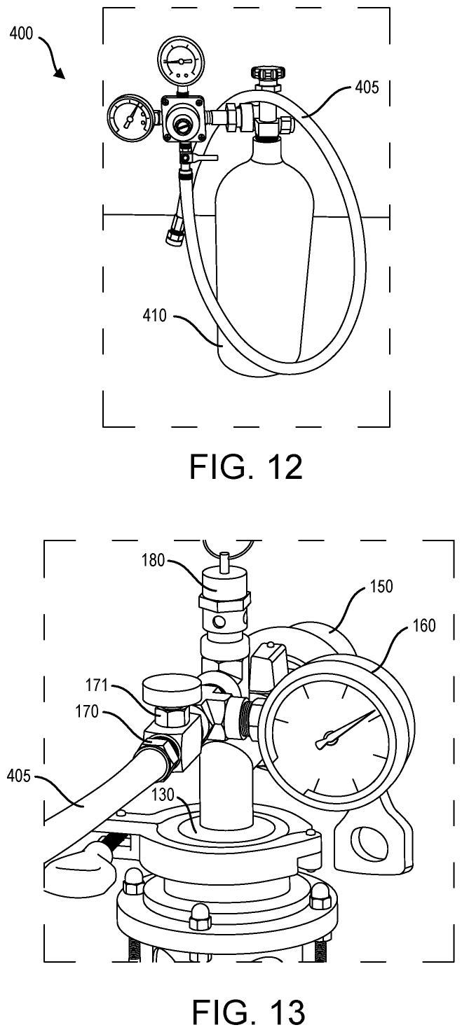

The apparatus 100 may include a fitting 170 that allows the vessel 105 to be fluidly connected to a supply of pressurized gas (e.g. nitrogen), a vacuum line for purging the vessel, or an output line connected to a ventilation system for expelling or recovering hydrocarbon gases from solvents. The apparatus 100 may include a valve 171 (e.g. a needle valve) located between the fitting 170 and the vessel 105. The fitting 170 may allow the vessel 105 to be fluidly connected to an input or output line. When the valve 171 is open, gas within the vessel 105 can be released through an output line (e.g. during purging of excess solvent gas). The valve 171 can then be closed and the output line disconnected. The fitting 170 may allow the vessel 105 to be fluidly connected to an external gas supply 400, as shown in FIG. 13. In one example, the external gas supply 400 may include a gas supply line 405 extending from a gas cylinder 410, as shown in FIG. 12. When the valve 171 is open, gas can be delivered from the gas supply line 405 to the vessel 105. Gas may be added to the vessel 105 until pressure within the vessel 105 is greater than atmospheric pressure. In one example, nitrogen gas may be added to the vessel 105 until pressure within the vessel 105 is about 10 to 12 psig. The valve 171 can then be closed and the gas supply line 405 disconnected. Providing a pressure above atmospheric pressure may promote growth of crystalline material within the vessel 105.

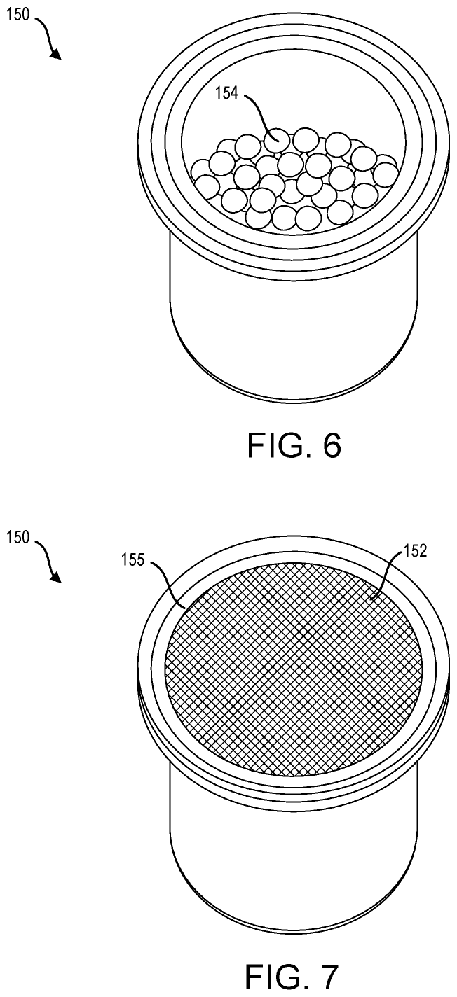

The apparatus 100 may include a desiccant chamber 150 fluidly connected to the vessel 105. The desiccant chamber 150 can be a container that is configured to receive a desiccant material 154, as shown in FIG. 6. The desiccant material 154 may be an FDA-approved, non-toxic desiccant. The desiccant material 154 may be a self-indicating desiccant. The desiccant material may include montmorillonite clay. The desiccant material may include silica gel. The desiccant material may include an indicating silica gel. The desiccant material may include calcium oxide. The desiccant material may include calcium sulfate.

In one example, the desiccant chamber 150 may be filled with desiccant material 154 to a level of about two-thirds full, as shown in FIG. 6. In another example, the desiccant chamber 150 can be filled with desiccant material 154 to a level between about one-half and about two-thirds full. In yet another example, the desiccant chamber 150 can be filled with desiccant material 154 to a level of at least one-quarter full.

After desiccant material 154 has been added to the desiccant chamber 150, a screen 152 may be placed over an opening in the desiccant chamber 150, as shown in FIG. 7. The screen 152 may retain the desiccant material 154 in the desiccant chamber 150 and prevent it from entering the vessel 105 and directly contacting the plant extract. The screen 152 may allow water vapor from the plant extract to pass from the vessel 105 to the desiccant chamber 150 where it can be captured by the desiccant material 154. The desiccant material 154 may non-invasively reduce the moisture content of the plant extract 190 and thereby promote crystal growth. In the example shown in FIG. 7, the screen 152 may be a screen gasket having a seal portion 155 extending around a perimeter of the screen. In another example, the screen 152 and seal portion 155 may be two separate components. In either instance, the seal portion 155 may be a chemical-resistant gasket. In one example, the gasket 155 may be a gasket made of a fluoropolymer elastomer, such as VITON.

Live resin 190 made from fresh cannabis material may contain a significant amount of water. Water may slow or inhibit crystal growth. It is therefore desirable to remove the water from the live resin during the crystallization process. The desiccant chamber 150 allows an operator to remove water from the live resin after the vessel 105 has been sealed. The desiccant material 154 removes water vapor from the live resin, which may increase the rate of crystal growth as well as the quality of the resulting crystals. The result is large, dense crystal clusters that are not possible without the desiccant chamber 150.

### Method of Producing HTFSE and HCFSE

The apparatus 100 is capable of producing High Terpene Full Spectrum Extract (HTFSE) and High Cannabinoid Full Spectrum Extract (HCFSE) from live resin. HCFSE may include crystalline tetrahydrocannabinolic acid (THCA), which falls out of the HTFSE solution. HTFSE may be a watery liquid containing 50-60% terpenes by weight. HTFSE may contain 20-40% THC by weight.

Cannabinoids and terpenes may be found in resin-filled glands, known as trichomes, that cover parts of cannabis plants. Cannabinoids and terpenes may be prevalent in upper leaves, flowers, and flower bracts of unfertilized female cannabis plants. Cannabinoids and terpenes may also be found on seed coverings and surrounding areas of pollinated plants.

Live resin can be produced by subjecting freshly cut or fresh, frozen cannabis plant material to an extraction process. The cannabis plant material used to make live resin may contain more than 0.3% THC on a dry weight basis. Prior to exposing fresh cannabis to the extraction process, the cannabis may be dried for about one day to reduce moisture content and then frozen using dry ice or liquid nitrogen. During the freezing step, the cannabis can be maintained at a temperature of about −50 to −60 degrees Fahrenheit or lower.

The frozen cannabis material can then be added to a closed loop extraction system or other suitable extraction system. Solvent used in the extraction process may be propane, butane, or a mixture of propane and butane. In a preferred example, the solvent mixture may be about 70% propane and about 30% butane. In another example, the solvent mixture may be greater than about 50% propane and less than about 50% butane. In yet another example, the solvent mixture may be less than about 50% propane and greater than about 50% butane. For safety, the solvent-based extraction should be performed in a certified Class 1, Division 1 (C1D1) space.

The extraction process may yield a cannabis extract that contains solvent. If necessary, excess solvent can be purged from a collection vessel 200 prior to transferring the extract to the apparatus vessel 105 while in a C1D1 space. Excess solvent can be purged using the closed loop extraction system. Alternately, heating the collection vessel 200 to a temperature of about 60 degrees Fahrenheit may promote vaporization and purging of the solvent without losing significant amounts of desirable, volatile terpenes. It is desirable to leave some solvent in the extract to reduce viscosity of the mixture and thereby allow it to be poured into the vessel 105, as shown in FIG. 9.

The vessel 105 may be maintained at a pressure above atmospheric pressure during the production of HTFSE and HCFSE. An elevated pressure within the vessel 105 may accelerate and/or promote formation of crystals. In a first example, where the live resin contains some hydrocarbon solvent, the vessel 105 may be naturally pressurized due to evaporation of the hydrocarbon solvent from the live resin after the vessel 105 is sealed. This process is referred to herein as “hydrocarbon pressurization.” A target vessel pressure for hydrocarbon pressurization may be about 4-6 psig. In a second example, where the live resin contains very little or no hydrocarbon solvent, the vessel 105 may be pressurized by connecting it to an external gas source 400. The gas source may supply nitrogen or another suitable gas. This process is referred to herein as “nitrogen pressurization.” A target vessel pressure for nitrogen pressurization may be about 10-12 psig.

The vessel 105 may be chilled prior to receiving the live resin 190. Chilling the vessel 105 may prevent the live resin 190 from foaming or boiling over during transfer. To chill the vessel 105, it may be placed in a freezer or exposed to liquid nitrogen prior to receiving the live resin 190.

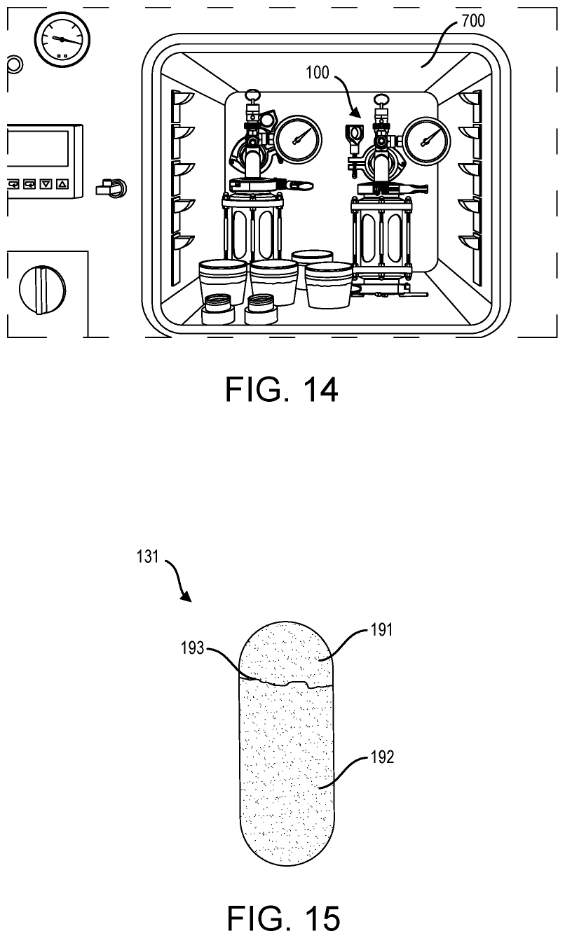

When using hydrocarbon pressurization, after the live resin 190 has been added to the vessel 105 and the vessel sealed, the vessel may be placed in a dark environment at room temperature. The pressure gauge 160 should be monitored. If the pressure within the vessel 105 exceeds the target pressure of 4-6 psig, the vessel 105 can be returned to the C1D1 room carefully to avoid agitating the live resin, and the needle valve 171 actuated to slowly decrease the pressure within the vessel 105 to a value between 4 and 6 psi. The vessel 105 can then be carefully returned to the dark, room-temperature environment. The vessel pressure can be monitored periodically (e.g. every 24 hours). If the pressure exceeds 6 psig, the above-described pressure relief process can be repeated as many times as needed to reduce the hydrocarbon solvent concentration within the vessel 105 to a target level. After the live resin 190 has settled and the vessel pressure has stabilized between about 4 and 6 psig (i.e. the hydrocarbon solvent concentration within the vessel 105 produces a pressure of about 4-6 psi), the sealed vessel 105 can be placed in a dark oven 700, as shown in FIG. 14, or other suitable, dark location at a temperature of about 75-80 degrees Fahrenheit, and preferably about 78 degrees Fahrenheit. The vessel 105 can remain in the oven 700 or other suitable, dark location for about 11-14 days without agitation. During this time, HCFSE and HTFSE will separate within the vessel 105. If the vessel 105 includes a side window 131, a separation line 193 will form between the HCFSE and the HTFSE, as shown in FIG. 15. The HCFSE will settle below the HTFSE.

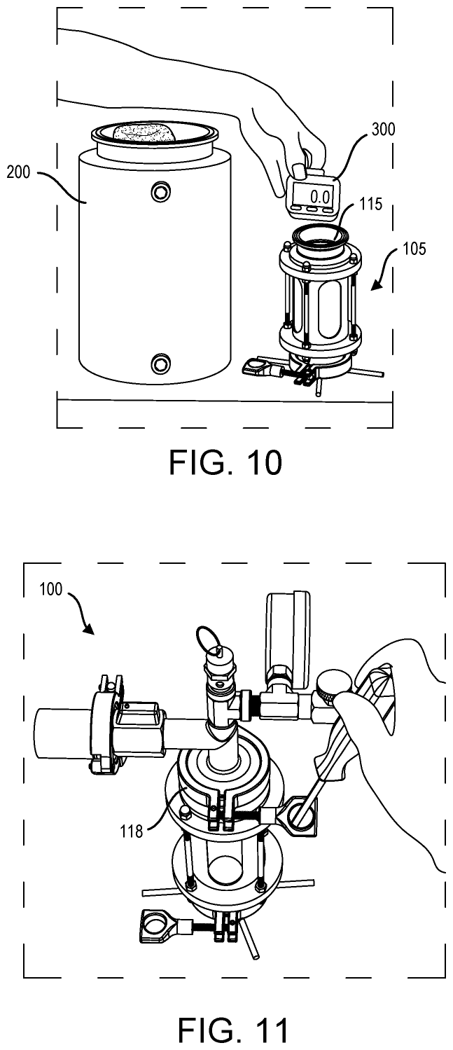

When using nitrogen pressurization, the live resin that is added to the vessel 105 may contain little or no solvent. Consequently, the sealed vessel 105 may be unable to achieve a pressure of 4-6 psi due to evaporation of solvent alone. As an alternative method for pressurizing the vessel 105, the vessel can be connected to an external gas source 400, such as an external nitrogen source, and slowly pressurized to about 10-12 psi using the needle valve 171. Pressurizing the vessel 105 with nitrogen may accelerate formation of crystalline material by about 2-3 days compared to hydrocarbon pressurization, but the resulting THCA crystals (also known as diamonds) may be smaller. After the live resin has been poured into the vessel 105, a hydrocarbon meter 300 can be used to measure hydrocarbon levels at the upper opening 115 of the vessel 105, as shown in FIG. 10. The hydrocarbon level should not exceed a lower explosive limit (LEL) of the solvent used in the extraction process. Ideally the hydrocarbon meter 300 will detect no hydrocarbons and display a reading of zero, as shown in FIG. 10. If the meter 300 detects hydrocarbons, the vessel 105 should remain open and at room temperature to permit vaporization and purging of any remaining solvent. After the meter 300 detects no hydrocarbons, the lid assembly 126 can be installed to seal the vessel 105, as shown in FIG. 11. After the live resin has been added to the vessel 105 and the vessel sealed, the vessel may be placed in a dark, refrigerated location for about 24-48 hours. The vessel 105 can then be moved carefully to a dark, room-temperature environment, avoiding agitation. The external gas supply 400 may be connected to the vessel 105 to deliver pressurized nitrogen. The gas supply line 405 extending from the gas cylinder 410 may be connected to the vessel 105 via the fitting 170. The valve may be slowly opened 171 to slowly add pressure to the vessel 105 until the target pressure of about 10-12 psi is reached. Adding nitrogen slowly to the vessel 105 may avoid agitation of the live resin. After the live resin 190 has settled and the vessel pressure has stabilized between 10 and 12 psig, the sealed vessel 105 can be placed in a dark oven 700, as shown in FIG. 14, or other suitable, dark location at a temperature of about 75-80 degrees Fahrenheit, and preferably about 78 degrees Fahrenheit. The vessel 105 can remain in the oven 700 or other suitable, dark location for about 7-12 days without agitation. During this time, HCFSE and HTFSE will separate within the vessel 105. If the vessel 105 includes a side window 131, a separation line 193 will form between the HCFSE and the HTFSE, as shown in FIG. 15. The HCFSE will settle below the HTFSE.

Regardless of whether the hydrocarbon pressurization process or the nitrogen pressurization process is used, the valve 151 for the desiccant chamber 150 can be opened to provide a fluid pathway 155 between the vessel 105 and the desiccant chamber 150. By opening the fluid pathway 155, the valve 151 can allow the desiccant material 154 to be exposed to and absorb water vapor from the live resin 190 and thereby reduce the water content of the live resin. Since water inhibits crystal growth, reducing water content of the live resin may promote crystal growth. The desiccant material 154 may absorb at least 10% of water contained in the live resin 190 after providing the fluid pathway 155 between the vessel 105 and the desiccant chamber 150. The desiccant material 154 may absorb at least 20% of water contained in the live resin 190 after providing the fluid pathway 155 between the vessel 105 and the desiccant chamber 150. The desiccant material 154 may absorb at least 40% of water contained in the live resin 190 after providing the fluid pathway 155 between the vessel 105 and the desiccant chamber 150. The desiccant material 154 may absorb at least 60% of water contained in the live resin 190 after providing the fluid pathway 155 between the vessel 105 and the desiccant chamber 150. The desiccant material 154 may absorb at least 80% of water contained in the live resin 190 after providing the fluid pathway 155 between the vessel 105 and the desiccant chamber 150. The desiccant material 154 may absorb at least 90% of water contained in the live resin 190 after providing the fluid pathway 155 between the vessel 105 and the desiccant chamber 150.

In addition to absorbing water, the desiccant material 154 may also absorb terpenes, flavonoids, and/or terpenoids. This can be undesirable, since these compounds may positively influence aroma and taste of the resulting product. Therefore, to minimize loss of terpenes, flavonoids, and/or terpenoids to the desiccant material, the valve 151 may be opened for a duration less than the total duration of the process. In one example, the valve 151 to the desiccant chamber 150 may be opened for less than about 24 hours beginning at the start of the heating step. In another example, the valve 151 to the desiccant chamber 150 may be opened for less than about 36 hours beginning at the start of the heating step. In yet another example, the valve 151 to the desiccant chamber 150 may be opened for less than about 48 hours during the crystallization process. Opening the valve 151 for a predetermined time and then closing the valve may allow a significant portion of water vapor to be absorbed by the desiccant material 154 while also limiting the amounts of terpenes, flavonoids, and/or terpenoids that are absorbed by the desiccant material.

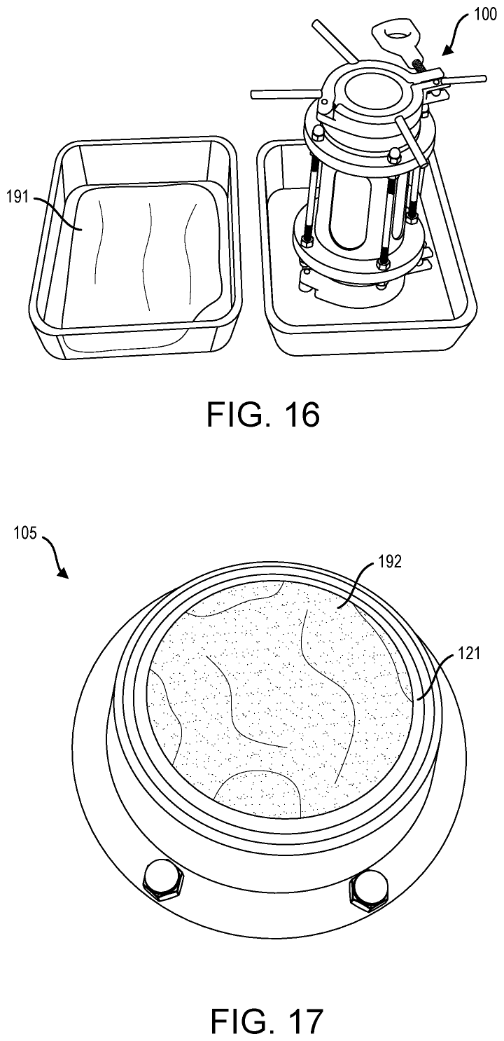

After the vessel 105 is removed from the oven 700, it can be returned to a C1D1 space and pressure within the vessel can be relieved through the fitting 170 by slowing opening the needle valve 171. After pressure has been relieved, the lid 130 can be removed from the vessel 105, and the HTFSE 191 can be poured from the vessel 105 into a suitable container, such as a PYREX dish, as shown in FIG. 16. The vessel 105 can then be positioned upside down in a suitable container for a period of about 12-24 hours to allow additional HTFSE 191 to drain from the vessel 105, as shown in FIG. 16. In one example, the collected HTFSE 191 can be heated to a temperature of about 110-120 degrees Fahrenheit (e.g. using a hot pad) for about 12 hours to purge any remaining solvent. In another example, the collected HTFSE 191 can then be heated to a temperature of about 95 degrees Fahrenheit in a vacuum oven for about 12 hours to purge any remaining solvent. Using a vacuum oven allows the final purge to occur at lower temperature, which can preserve desirable terpenes.

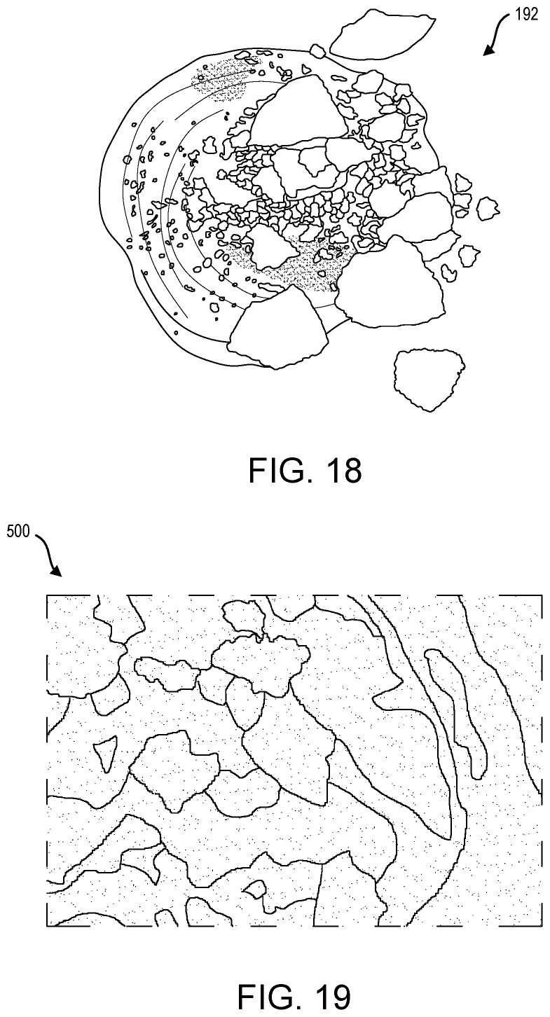

After the HTFSE 191 has been drained from the vessel 105, the bottom wall 107 of the vessel 105 can be removed to reveal the HCFSE 192, as shown in FIG. 17. The HCFSE 192 can then be removed from the vessel 105. If hydrocarbon pressurization is used, the HCFSE 192 will typically break into large crystal clusters, as shown in FIG. 18. If nitrogen pressurization is used, the HCFSE 192 will typically pour out as small sand-sized crystals. After the HTFSE and HCFSE have been removed from the vessel 105, the vessel can be cleaned with ethanol or isopropyl alcohol.

### Method of Producing THCA Sugar

The apparatus 100 is capable of producing THCA sugar from crude oil extracted from dried and cured cannabis plant material. A method for producing THC sugar may be similar to the method described above for producing HTFSE and HCFSE, however, the input material (i.e. plant extract) may be made from dried plant material instead of fresh or fresh, frozen plant material. The cannabis plant material may contain more than 0.3% THC on a dry weight basis. Oil extracted from dried and cured cannabis may contain more wax than oil extracted from fresh plant material. The presence of wax may inhibit separation of crystals from terpenes, resulting in a sugar-like product with terpenes infused into or otherwise mixed with the THCA crystal structures.

The extraction process may yield a cannabis extract (i.e. crude oil) that contains solvent. The solvent can be purged from a collection vessel 200 prior to transferring the extract to the vessel 105 while in a C1D1 space. Heating the collection vessel 200 to a temperature of about 60 degrees Fahrenheit may promote vaporization and purging of the solvent without losing significant amounts of desirable, volatile terpenes. Some solvent may be left in the extract to reduce viscosity of the mixture and thereby allow it to be poured into the vessel 105, as shown in FIG. 9.

The vessel 105 may be maintained at a pressure above atmospheric pressure during the production of THCA sugar. An elevated pressure within the vessel 105 may accelerate and/or promote formation of crystals. In a first example, where the crude oil contains some hydrocarbon solvent, the vessel 105 may be naturally pressurized by evaporation of the hydrocarbon solvent from the crude oil after the vessel 105 is sealed. This process is referred to herein as “hydrocarbon pressurization.” A target vessel pressure for hydrocarbon pressurization may be about 4-6 psig. In a second example, where the crude oil contains very little or no hydrocarbon solvent, the vessel 105 may be pressurized by connecting it to an external gas source 400. The gas may be nitrogen or another suitable gas. This process is referred to herein as “nitrogen pressurization.” A target vessel pressure for nitrogen pressurization may be about 10-12 psig.

The vessel 105 may be chilled prior to receiving the crude oil. Chilling the vessel 105 may prevent the crude oil from foaming or boiling over during transfer. To chill the vessel 105, it may be placed in a freezer or exposed to liquid nitrogen prior to receiving the crude oil.

When using hydrocarbon pressurization, after the crude oil has been added to the vessel 105 and the vessel sealed, the vessel may be placed in a dark environment at room temperature. The pressure gauge 160 should be monitored. If the pressure within the vessel 105 exceeds the target pressure of 4-6 psig, the vessel 105 can be returned to the C1D1 room carefully to avoid agitating the crude oil, and the needle valve 171 actuated to slowly decrease the pressure within the vessel 105 to a value between 4 and 6 psi. The vessel 105 can then be carefully returned to the dark, room temperature environment. The vessel pressure can be monitored periodically (e.g. every 24 hours). If the pressure exceeds 6 psig, the above-described pressure relief process can be repeated as many times as needed to reduce the hydrocarbon solvent concentration within the vessel 105. After the crude oil has settled and the vessel pressure has stabilized between about 4 and 6 psig (i.e. the hydrocarbon solvent concentration within the vessel 105 produces a pressure of about 4-6 psi), the sealed vessel 105 can be placed in a dark oven 700, as shown in FIG. 14, or other suitable, dark location at a temperature of about 75-80 degrees Fahrenheit, and preferably about 78 degrees Fahrenheit. The vessel 105 can remain in the oven 700 or other suitable, dark location for about 4-5 days without agitation. During this time, THCA sugar will from in the vessel 105.

When using nitrogen pressurization, the crude oil may contain little or no solvent. Consequently, the vessel 105 may be unable to achieve a pressure of 4-6 psi due to evaporation of solvent alone. As an alternative method for pressurizing the vessel 105, it can be connected to an external gas source 400, such as an external nitrogen source, and slowly pressurized to about 10-12 psi using the needle valve 171. Pressurizing the vessel 105 with nitrogen may accelerate formation of crystalline material compared to hydrocarbon pressurization, but the resulting THCA crystals (i.e. diamonds) may be smaller. After the crude oil has been poured into the vessel 105, a hydrocarbon meter 300 can be used to measure hydrocarbon levels at the upper opening 115 of the vessel 105, as shown in FIG. 10. The hydrocarbon level should not exceed a lower explosive limit of the solvent used in the extraction process. Ideally the hydrocarbon meter will detect no hydrocarbons and display a reading of zero. If the meter 300 detects hydrocarbons, the vessel 105 should remain open and at room temperature to permit vaporization and purging of any remaining solvent. After the meter 300 detects no hydrocarbons, the lid assembly 126 can be installed to seal the vessel 105, as shown in FIG. 11. After the crude oil has been added to the vessel 105 and the vessel sealed, the vessel may be placed in a dark, refrigerated location for about 24-48 hours. The vessel 105 can then be moved carefully to a dark, room-temperature environment, avoiding agitation. The external gas supply 400 may be connected to the vessel 105 to deliver pressurized nitrogen. The gas supply line 405 extending from the gas cylinder 410 may be connected to the vessel 105 via the fitting 170. The valve may be slowly opened 171 to slowly add pressure to the vessel 105 until the target pressure of about 10-12 psi is reached. Adding nitrogen slowly to the vessel 105 may avoid agitating the crude oil. After the crude oil has settled and the vessel pressure has stabilized between 10 and 12 psig, the sealed vessel 105 can be placed in a dark oven 700, as shown in FIG. 14, or other suitable, dark location at a temperature of about 75-80 degrees Fahrenheit, and preferably about 78 degrees Fahrenheit. The vessel 105 can remain in the oven 700 or other suitable, dark location for about 4-5 days without agitation. During this time, THCA will form within the vessel 105.

Regardless of whether the hydrocarbon pressurization process or the nitrogen pressurization process is used, the valve 151 for the desiccant chamber 150 can be opened to provide a fluid pathway 155 between the vessel 105 and the desiccant chamber 150. By opening the fluid pathway 155, the valve 151 can allow the desiccant material 154 to be exposed to and absorb water vapor from the crude oil 190 and thereby reduce the water content of the crude oil. Since water inhibits crystal growth, reducing water content of the crude oil may promote crystal growth. The desiccant material 154 may absorb at least 10% of water contained in the crude oil 190 after providing the fluid pathway 155 between the vessel 105 and the desiccant chamber 150. The desiccant material 154 may absorb at least 20% of water contained in the crude oil 190 after providing the fluid pathway 155 between the vessel 105 and the desiccant chamber 150. The desiccant material 154 may absorb at least 40% of water contained in the crude oil 190 after providing the fluid pathway 155 between the vessel 105 and the desiccant chamber 150. The desiccant material 154 may absorb at least 60% of water contained in the crude oil 190 after providing the fluid pathway 155 between the vessel 105 and the desiccant chamber 150. The desiccant material 154 may absorb at least 80% of water contained in the crude oil 190 after providing the fluid pathway 155 between the vessel 105 and the desiccant chamber 150. The desiccant material 154 may absorb at least 90% of water contained in the crude oil 190 after providing the fluid pathway 155 between the vessel 105 and the desiccant chamber 150.

In addition to absorbing water, the desiccant material 154 may also absorb terpenes, flavonoids, and/or terpenoids. This can be undesirable, since these compounds may influence aroma and taste of the resulting product. Therefore, to minimize loss of terpenes, flavonoids, and/or terpenoids to the desiccant material, the valve 151 may be opened for a duration less than the total duration of the process. In one example, the valve 151 to the desiccant chamber 150 may be opened for less than about 24 hours beginning at the start of the heating step. In another example, the valve 151 to the desiccant chamber 150 may be opened for less than about 36 hours beginning at the start of the heating step. In yet another example, the valve 151 to the desiccant chamber 150 may be opened for less than about 48 hours during the crystallization process. Opening the valve 151 for a predetermined time and then closing the valve may allow a significant portion of water vapor to be absorbed by the desiccant material 154 while also limiting the amounts of terpenes, flavonoids, and/or terpenoids that are absorbed by the desiccant material.

After the vessel 105 is removed from the oven 700, it can be returned to a C1D1 space and pressure within the vessel can be relieved through the fitting 170 by slowing opening the needle valve 171. After pressure has been relieved, the lid 130 can be removed from the vessel 105, and the THCA sugar can be scraped from the vessel 105 into a suitable container, such as a PYREX dish. The resulting THCA sugar 500, shown in FIG. 19, may include a mixture of THCA crystals and terpenes. The collected THCA sugar 500 can be heated to a temperature of about 110-120 degrees Fahrenheit (e.g. using a hot pad) for about 12 hours to purge any remaining solvent. Alternately, the collected THCA sugar 500 can be heated to a temperature of about 95 degrees Fahrenheit in a vacuum oven for about 12 hours to purge any remaining solvent. Using a vacuum oven allows the final purge to occur at lower temperature, which may preserve desirable terpenes. After the THCA crystals 500 have been removed from the vessel 105, the vessel can be cleaned with ethanol or isopropyl alcohol.

### Method of Producing CBD Crystals

A mature cannabis plant can contain hundreds of cannabinoids, terpenes, and flavonoids. When creating cannabis concentrates, the cannabinoids, terpenes, and flavonoids can be extracted from the cannabis plant material though various extraction processes. To refine these compounds into their purest form, a distillation process can be performed. Distillation is an extraction process that separates and refines cannabinoids at a molecular level. Short path fractional distillation may produce single compound oils that can have purities of about 99% or higher.

The apparatus 100 can be used to produce CBD crystals from a suitable cannabis oil distillate. An example of a suitable single compound oil derived from cannabis plant material is CBD distillate. CBD distillate can be made from CBD crude oil. To obtain CBD distillate from CBD crude oil, the crude oil may be winterized, decarboxylated, and/or distilled. The purpose of the winterization process is to remove waxes, fats, and other lipids from the oil. The purpose of the decarboxylation process is to convert cannabidiolic acid (CBDA) in the oil to cannabidiol (CBD). CBD's molecular structure lends itself to formation of crystalline, whereas CBDA's molecular structure may not. CBDA is a non-psychoactive cannabinoid and a precursor to CBD. Decarboxylation may occur when the oil is exposed to heat or sunlight. CBDA is present in hemp plants, which may be bred to cultivate higher CBDA levels.

Crude cannabis extract may be distilled by heating the CBD to its boiling point. CBD vapor may condense and be collected in a separate container and undesirable components, such as chlorophyll, may remain in the original container. Residual solvent used during an initial extraction process may be removed during distillation resulting in a CBD distillate that is substantially solvent free. CBD distillate can be made from hemp plants. Hemp plants may contain less than or equal to 0.3% THC on a dry weight basis.

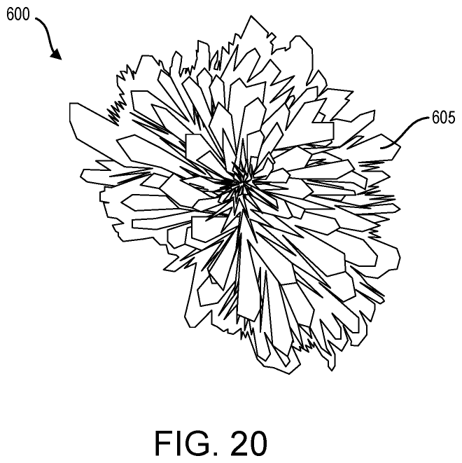

A method for producing CBD crystals may include mixing the cannabis distillate (e.g. CBD distillate) with an organic compound. In one example, the organic compound can be n-pentane. The CBD distillate can be mixed with the organic compound at room temperature until the distillate fully dissolves. The mixture can then be poured into the vessel 105. The vessel 105 can be sealed and placed in a refrigerator for a period of time (e.g. overnight). The valve 151 for the desiccant chamber 150 can be opened to allow the desiccant material 154 to absorb water vapor from the atmosphere (e.g. air) within the vessel 105. Since water inhibits crystal growth, reducing water content of the atmosphere within the vessel 105 may promote crystal growth. CBD crystals 600, such as those shown in FIG. 20, will grow within the vessel 105 while it is being refrigerated.

The desiccant material 154 may absorb at least 10% of water contained in the vessel atmosphere after providing the fluid pathway 155 between the vessel 105 and the desiccant chamber 150. The desiccant material 154 may absorb at least 20% of water contained in the vessel atmosphere after providing the fluid pathway 155 between the vessel 105 and the desiccant chamber 150. The desiccant material 154 may absorb at least 40% of water contained in the vessel atmosphere after providing the fluid pathway 155 between the vessel 105 and the desiccant chamber 150. The desiccant material 154 may absorb at least 60% of water contained in the vessel atmosphere after providing the fluid pathway 155 between the vessel 105 and the desiccant chamber 150. The desiccant material 154 may absorb at least 80% of water contained in the vessel atmosphere after providing the fluid pathway 155 between the vessel 105 and the desiccant chamber 150. The desiccant material 154 may absorb at least 90% of water contained in the vessel atmosphere after providing the fluid pathway 155 between the vessel 105 and the desiccant chamber 150. Typically, CBD distillate will be dewatered through the distillation process. However, if the CBD distillate somehow absorbs water prior to being added to the vessel 105, the desiccant material 154 can absorb all or a portion of the water and thereby promote growth of CBD crystals.

After the refrigeration step, the CBD crystals 600 can be removed from the vessel 105. The CBD crystals may contain over 90% pure CBD in crystalline form. The CBD crystals may contain over 95% pure CBD in crystalline form. The CBD crystals may contain over 98% pure CBD in crystalline form. The CBD crystals may contain over 99% pure CBD in crystalline form. The CBD crystals may contain about 96%-99.9% pure CBD in crystalline form. In one example, purity testing of the resulting CBD isolate may be performed using Nuclear Magnetic Resonance (NMR) testing. In another example, purity testing of the resulting CBD isolate may be performed using High-Pressure Liquid Chromatography (HPLC).

In one example, about 50 grams of CBD distillate can be placed in the vessel 105 of the apparatus 100. The vessel 105 may have an inner volume 110 about equal to 300 mL (10.14 oz), an inner width (w) of about 2 inches, and an inner depth (d) of about 3.2 inches. In this example, the depth (d) is about 1.6 times greater than the width (w). A ratio of about 0.5:1 to about 3:1 by mass of pure n-pentane at room temperature can be added to the CBD distillate. In one example, 150 grams of pentane can be added to the 50 grams of CBD distillate to provide a 3:1 ratio by mass. The CBD distillate can then be mixed with the pentane at room temperature until the distillate fully dissolves. The mixture can then be poured into the vessel 105. The vessel 105 can be sealed and placed in a refrigerator at about 30-35 degrees Fahrenheit for about 12-36 hours. CBD crystals 600, such as those shown in FIG. 20, will grow within the vessel 105 while it is being refrigerated. The vessel 105 can then be opened and the pentane poured out. The CBD crystals 600 can then be removed from the vessel 105.

In one example, about 100 grams of CBD distillate can be placed in the vessel 105 of the apparatus 100. The vessel 105 may have an inner volume about equal to 700 mL (23.67 oz), an inner diameter (w) of about 3 inches, and an inner depth (d) of about 3.3 inches. In this example, the depth (d) is about 1.1 times greater than the width (w). A ratio of about 0.5:1 to about 3:1 by mass of pure n-pentane at room temperature can be added to the CBD distillate. In one example, 200 grams of pentane can be added to the 100 grams of CBD distillate to provide a 2:1 ration by mass. The CBD distillate can be mixed with the pentane at room temperature until the distillate fully dissolves. The mixture can then be poured into the vessel 105. The vessel 105 can be sealed and placed in a refrigerator at about 30-35 degrees Fahrenheit for about 12-36 hours. CBD crystals 600, such as those shown in FIG. 20, will grow within the vessel 105 while it is being refrigerated. The vessel 105 can then be opened and the pentane poured out. The CBD crystals 600 can then be removed from the vessel 105.

The elements and method steps described herein can be used in any combination whether explicitly described or not. All combinations of method steps as described herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

The methods and compositions of the present invention can comprise, consist of, or consist essentially of the essential elements and limitations described herein, as well as any additional or optional steps, components, or limitations described herein or otherwise useful in the art.

It is understood that the invention is not confined to the particular construction and arrangement of parts herein illustrated and described, but embraces such modified forms thereof as come within the scope of the claims.

The foregoing description has been presented for purposes of illustration and description. It is not intended to be exhaustive or to limit the claims to the embodiments disclosed. Other modifications and variations may be possible in view of the above teachings. The embodiments were chosen and described to explain the principles of the invention and its practical application to enable others skilled in the art to best utilize the invention in various embodiments and various modifications as are suited to the particular use contemplated. It is intended that the claims be construed to include other alternative embodiments of the invention except insofar as limited by the prior art.

What is claimed is:

1. An apparatus for producing crystalline material from plant extract, the apparatus comprising:
a vessel comprising an inner volume defined by one or more inner surfaces, the inner volume configured to receive a plant extract;
a desiccant chamber fluidly connected to the vessel, the desiccant chamber configured to receive a desiccant material;
a fluid pathway extending from the inner volume of the vessel to the desiccant chamber; and
a valve located in the fluid pathway, the valve having an open position and a closed position, wherein water vapor can travel through the fluid pathway from the inner volume to the desiccant chamber when the valve is in the open position.

2. The apparatus of claim 1, further comprising a screen in the fluid pathway and covering an opening in the desiccant chamber, the screen configured to retain a desiccant material in the desiccant chamber.

3. The apparatus of claim 1, further comprising a desiccant material in the desiccant chamber, wherein the desiccant material comprises an indicating silica gel.

4. The apparatus of claim 1, further comprising a desiccant material in the desiccant chamber, wherein the desiccant material comprises montmorillonite clay, silica gel, indicating silica gel, calcium oxide, or calcium sulfate.

5. The apparatus of claim 1, wherein the inner volume has a maximum inner depth and a maximum inner width, the maximum inner depth being greater than the maximum inner width.

6. The apparatus of claim 1, further comprising a plurality of nucleation sites on the one or more inner surfaces of the vessel.

7. The apparatus of claim 1, wherein the plant extract is cannabis extract and the crystalline material comprises cannabidiol crystals comprising over 95% pure cannabidiol.

8. An apparatus for producing crystalline material from a cannabis plant extract, the apparatus comprising:
a vessel for receiving the cannabis plant extract, the vessel comprising an inner surface, an outer surface, and an opening, the inner surface defining an inner volume;
a lid assembly sealing the opening of the vessel, the lid assembly comprising a desiccant chamber fluidly connected to the lid assembly, the desiccant chamber configured to receive a desiccant material; and
a fluid pathway extending from the inner volume of the vessel to the desiccant chamber,
wherein water vapor can travel through the fluid pathway from the inner volume to the desiccant chamber.

9. The apparatus of claim 8, further comprising a valve in the fluid pathway, the valve having an open position and a closed position, wherein water vapor can travel through the fluid pathway from the inner volume to the desiccant chamber when the valve is in the open position.

10. The apparatus of claim 8, further comprising a desiccant material in the desiccant chamber.

11. The apparatus of claim 8, wherein the inner volume has a maximum depth and a maximum width, the maximum depth being at least 1.6 times greater than the maximum width.

12. The apparatus of claim 8, wherein the inner surface comprises one or more laser etched nucleation sites.

13. The apparatus of claim 8, wherein the cannabis plant extract is a hemp-derived cannabis plant extract.

14. The apparatus of claim 8, wherein the desiccant chamber is a removable desiccant chamber located above a top wall of the vessel.

15. A method of manufacturing crystalline cannabidiol material from cannabidiol distillate, the method comprising:
dissolving a cannabidiol distillate in a liquid organic compound to form a mixture;
placing the mixture in an inner volume of a vessel;
fluidly connecting the inner volume of the vessel to a desiccant chamber containing a desiccant material;
sealing the vessel, the vessel containing air and the mixture;
exposing the desiccant material to the air in the vessel by providing a fluid pathway between the vessel and the desiccant chamber; and
refrigerating the vessel for a period of time to allow crystalline cannabidiol material to form from the mixture.

16. The method of claim 15, wherein the liquid organic compound is liquid pentane.

17. The method of claim 15, wherein refrigerating the vessel for a period of time comprises refrigerating the vessel for about 12 to 36 hours at a temperature of about 32 degrees Fahrenheit.

18. The method of claim 15, wherein mixing a cannabidiol distillate with a liquid organic compound to form a mixture comprises mixing about 0.5 to 3 parts pentane with 1 part cannabidiol distillate by mass.

19. The method of claim 15, further comprising allowing the desiccant material to absorb at least 40% of water contained in the air after providing a fluid pathway between the vessel and the desiccant chamber.

20. The method of claim 15, wherein the crystalline cannabidiol material comprises cannabidiol crystals comprising over 95% pure cannabidiol.
